(19) United States
(12) Patent Application Publication
(10) Pub. No.: US 2012/0295313 A1
(43) Pub. Date: Nov. 22, 2012

(54) PROCESS FOR PRODUCING GRANULES

(75) Inventors: Lars Berglund, Lulea (SE); Houssine Sehaqui, Solna (SE)

(73) Assignee: SweTree Technologies AB, Umeå (SE)

(21) Appl. No.: 13/508,715

(22) PCT Filed: Nov. 11, 2010
(86) PCT No.: PCT/SE2010/051239

§ 371 Date: Jul. 26, 2012

Related U.S. Application Data
(60) Provisional application No. 61/260,861, filed on Nov. 13, 2009.

(30) Foreign Application Priority Data
Nov. 13, 2009 (SE) 0950858-1

(51) Int. Cl. C08L 5/06; B29B 9/06; C12P 19/04

(52) U.S. Cl. 435/101; 106/162.9; 264/141; 264/297.2

(57) ABSTRACT

The present invention pertains to processes for preparing granules comprising cellulose-containing fibers and biocomposites comprising disintegrated fibers, granules and biocomposites produced by the processes of the invention, as well as uses of said granules in methods for producing biocomposites comprising disintegrated fibers.

### FIELD OF THE INVENTION

The present invention pertains to processes for preparing granules comprising cellulose-containing fibers and biocomposites, granules produced by the process of the invention, as well as uses of said granules in methods for producing biocomposites comprising disintegrated fibers.

### TECHNICAL BACKGROUND

Biocomposites, the green alternative to ordinary composites derived from petrochemicals, generally consists of a biodegradable polymer as a matrix material and a biodegradable filler as reinforcement material. Starch is one of the most promising polysaccharide polymers for replacing petroleum-based plastics and it is commonly utilized as a matrix polymer, as a result of its desirable characteristics in terms of abundance and biodegradability. In addition, when starch is mixed with water and another plasticizer, typically glycerol, it can be melt-processed as thermoplastic starch (TPS). In spite of its appealing physical and chemical features, numerous complicating issues arise when utilizing starch in commercial production.

Unlike other carbohydrates and edible polymers, starch occurs as discrete semi-crystalline hydrophilic particles, composed of amylose and amylopectin, called starch granules. The amylose component has a high melt viscosity which negatively affects its processability. Additionally, starch is hygroscopic and sensitive to ageing, leading to undesirable structural changes and decrystallization during long-term storage of starch-containing products, resulting in inferior properties. Hence, conventional processing techniques are generally unsuitable for starch as thermal degradation occurs before the polymer enters it melting state. However, utilizing specific combinations of high temperature, shear, and water, starch can be processed like a thermoplastic material, albeit still displaying a very narrow processing window. With the addition of various types of plasticizers, the physical and chemical properties of the resulting TPS material can be modulated.

Biodegradable fillers utilized as biocomposite reinforcement material are relatively frequently derived from cellulose, the linear β-(1,4)-glucan that constitutes the major part of the plant cell wall. Cellulosic fibers are commonly utilized for numerous applications within virtually any technical field, not only limited to the paper, pulp, and textile industries, but applications are also frequently found within the pharmaceutical sciences and within the process industry. Accordingly, the physical and chemical properties of cellulose have been the foci of substantial research efforts, and the structural composition and the characteristics of this polysaccharide is well known. The plant cell wall comprises cellulose fibrils, which in turn are composed of nanofibrils deriving from individual cellulose chains. The nanofibrils possess tremendously attractive mechanical properties and their high aspect ratio (with a length up to several micrometers and a width in the order of 5-100 nm) makes the polymer a promising substitute for synthetic fibers. In comparison to traditional cellulosic fibers, the most important advantage associated with nanosized fibers (i.e. nanofibrils) as filler/reinforcement in a composite material is the improved mechanical properties, even at a low filler content.

Extracting cellulosic nanofibrils is an inherently difficult process based on mechanical disintegration of the plant cell wall. Despite applying significant mechanical force and various chemical pre-treatments, such as strong hydrolysis, the disintegration process almost always results in bundles of nanofibrils, and not the highly desired individualized nanofibrils (Saito et al., Communications, Biomacromolecules, Vol. 7, 2006).

Melt processing and other conventional processing routes are mainstay workhorses for most industries, notably the automotive industry and the furniture industry. Composites, and in particular nanocomposites, constitute an attractive starting material for melt processing. However, nanocomposites suffer from high melt viscosity, with the implication that the filler content becomes unacceptably low. The reason for this is the fact that the high aspect ratio and large specific surface area of the nanofibers cause high melt viscosity. Further, commonly utilized processing conditions, aimed at enabling feasible reaction systems, further imply that the nanofiber content is generally very low, i.e. approximately 0.5%, a content that does not significantly improve the properties of the resulting composites.

Solvent casting, for instance, is primarily a laboratory method, suffering from inter alia flocculation problems, implying that its industrial relevance is currently limited (Svagan et al., Biomacromolecules, Vol 8, 2007).

Other methods for producing cellulose nanocomposites include impregnation of dry cellulose nanofiber networks (Nakagaito et al., Applied Physics A, Vol 80, 2005) and compression moulding of dry cellulose nanofiber networks between two thermoplastic sheets; two methods with inherent problems.

### SUMMARY OF THE INVENTION

It is thus an object of the present invention to solve the above-identified problems and satisfy the existing needs, i.e., firstly, to enable the use of an inexpensive biocomposite as an efficient disintegration environment for conventional processing techniques, despite the undesirable characteristics of the most common matrix polymer (starch), secondly, to allow for obtaining cellulosic nanocomposite material comprising a high proportion of microfibrillated cellulose from cellulose-containing fibers, wherein the nanocomposite material possesses an increased strength and stiffness compared to the current art, and, thirdly, to provide a novel technique for preparing granules, enabling manufacture of nanocomposites with homogenously dispersed nanofibers present within the polymer matrix. Hence, the present invention pertains to a process for preparing granules comprising cellulose-containing fibers, in order to enable further processing of said granules into desirable (nanocomposite) products.

More specifically, the process of the present invention comprises the steps of pre-treating cellulose-containing fibers, optionally washing and filtrating the pre-treated cellulose-containing fibers in order to remove pre-treatment agents and excess solvent, adding at least one type thermoplastic polymer to the pre-treated cellulose-containing fibers, and, finally, compounding the mixture obtained in the preceding step, thereby obtaining said granules. Further, granules comprising between approximately 5 and 70% pre-treated cellulose-containing fibers and between approximately 30 and 95% of at least one thermoplastic polymer are also within the scope of present invention. Additionally, the present invention relates to granules obtainable by the above process, and to the use of the granules of the present invention for producing disintegrated nanofibers and composite materials with superior properties.

The present invention enables efficient, fast, and simplified production of granules for manufacture of pulp-derived composites comprising cellulose-containing fibers, using an optimized process carried out without excessive mechanical force and at moderate temperatures. Further, partially as an implication of the above, the invention permits using a disintegration environment comprising solely environmentally friendly biocomposite components, utilizing conventional processing techniques such as for instance melt processing. Additionally, as the major disintegration of the fibers comprised in the granules is carried out during the processing step, the problem of increasing viscosity is eliminated, as the increase in viscosity does not occur until the very last processing step. This is in contrast to the current art, wherein, firstly, petrochemically derived products are almost always utilized as matrix and filler, and, secondly, where the increase in melt viscosity implies that utilizing nanocomposites based on microfibrillated cellulose is highly unpractical, if not impossible. Moreover, the granules of the present invention constitute a versatile starting material for production of composites comprising microfibrillated cellulose in a thermoplastic matrix. The constituents of the granules, as well as the method of manufacturing the granules, imply that long-term storage is possible, which is highly advantageous for shipping and processing purposes. Furthermore, the resulting microfibrillated cellulose in the final biocomposite obtained from the granules possesses numerous desirable properties, such as for instance increased stiffness and strength. Additionally, the ease of processing, the scalable process per se, as well as the scalable individual steps, allow for rapid and robust scale up for the manufacturing of composite material.

### BRIEF DESCRIPTION OF THE DRAWINGS

FIG. 1. Stress-strain curves of samples with 0 and 6 wt % fibers (a) and 0 and 20 wt % fibers (b).

FIG. 2. Evolution of stiffness versus fiber content for composites with conventional cellulose-containing fibers and treated cellulose-containing fibers. Extrusion with a screw speed of 40 rpm. (▪) Represents TPS+Tempo treated fibers and (▴) represents TPS+pulp fibers.

FIG. 3. Evolution of tensile strength according to the fiber content for composites with conventional cellulose-containing fibers and treated cellulose-containing fibers. Extrusion with a screw speed of 40 rpm. (▪) Represents TPS+Tempo treated fibers and () represents TPS+pulp fibers.

FIG. 4: Stress-strain curves of composites with 6 wt % of treated or untreated fibers at two different screw speeds 40 and 80 rpm. 1=TPS+6% TEMPO-treated pulp fibers, 40 rpm; 2=TPS+6% TEMPO-treated pulp fibers, 80 rpm; 3=TPS+6% pulp fibers, 40 rpm; 4=TPS+6% pulp fibers, 80 rpm

FIG. 5: Stress-strain curves of composites containing 20 wt % of untreated or treated fibers for two different screw speeds 40 and 80 rpm. 1=TPS+20% TEMPO-treated pulp fibers, 40 rpm; 2=TPS+20% TEMPO-treated pulp fibers, 80 rpm; 3=TPS+20% pulp fibers, 80 rpm; 4=TPS+20% pulp fibers, 40 rpm

FIG. 6: Evolution of Young's Modulus (a) and tensile strength (b) according to fibers content in composites containing treated cellulose-containing fibers. In 6A (▪) Represents TPS+TEMPO treated fibers 40 rpm and (◯) represents TPS+TEMPO treated fibers 80 rpm. In 6B (□) Represents TPS+TEMPO treated fibers 40 rpm and () represents TPS+TEMPO treated fibers 80 rpm.

FIG. 7: Evolution of Young's Modulus (a) and tensile strength (b) according to fibers content in composites containing original cellulose-containing fibers. In 7A (▪) Represents TPS+Pulp fibers 40 rpm and (◯) represents TPS+Pulp fibers, 80 rpm. In 7B (□) Represents TPS+Pulp fibers 40 rpm and () represents TPS+Pulp fibers, 80 rpm.

FIG. 8: TGA curve of the TPS matrix and its first derivative.

FIG. 9: TGA curve of the composite TPS+20% PF (pure fibers?) and its first derivative.

FIG. 10: TGA curve of the composite TPS+20% TempoF and its first derivative.

FIG. 11: TGA curves comparison for composites with 20 wt % of cellulose-containing fibers and treated cellulose-containing fibers. 1=TPS+TEMPO-treated pulp fibers, 20%; 2=TPS+pulp fibers, 20%; 3=Pure TPS.

FIG. 12: DMA curves of the neat TPS matrix, and the two types of composites with 20% of untreated and treated fibers, respectively. 1=TPS+TEMPO-treated pulp fibers, 20%; 2=TPS+pulp fibers, 20%; 3=Pure TPS.

FIG. 13: SEM images of composites with 6% of fibers (T=treated or U=untreated) processed at 40 rpm at low magnification (×200).

FIG. 14: SEM images of the composite with 20% of TEMPO treated fibers processed at 40 rpm at two different magnifications (×100 and ×350)

FIG. 15: SEM images of composites with TPS and 20% of untreated fibers processed at 40 rpm and 80 rpm at low magnification (×100).

FIG. 16: SEM images of the composite TPS+20% treated fibers processed at 40 rpm at different magnification (×45000 and ×100000).

FIG. 17: Stress-strain curves of TPS and composites with treated fibers obtained after conditioning of samples at 5% RH and 50% RH for at least 10 days. 1=TPS at 5% RH; 2=TPS+20% Treated fibers at 50% RH; 3=TPS+20% Treated fibers at 5% RH; 4=TPS+6% Treated fibers at 5% RH; 5=TPS+6% Treated fibers at 50% RH; 6=TPS at 50% RH.

FIG. 18: Stress-strain curves of TPS and composites with untreated fibers obtained after conditioning of samples at 5% RH and 50% RH for at least 10 days. 1=TPS at 5% RH; 2=TPS+6% Treated fibers at 5% RH; 3=TPS+20% Treated fibers at 5% RH; 4=TPS+20% Treated fibers at 50% RH; 5=TPS+6% Treated fibers at 50% RH; 6=TPS at 50% RH.

### DETAILED DESCRIPTION OF THE INVENTION

The present invention relates to a process for preparing granules comprising cellulose-containing fibers, wherein the granules enable subsequent production of composites comprising individualized cellulose-derived microfibrillated cellulose (MFC). The invention further pertains to the granules per se, as well as the use of the granules for various purposes relating to disintegration of MFC from cellulose-containing fibers in the cellulose-containing fiber/polymer mixture.

Where features, embodiments, or aspects of the present invention are described in terms of Markush groups, a person skilled in the art will recognize that the invention may also thereby be described in terms of any individual member or subgroup of members of the Markush group. The person skilled in the art will further recognize that the invention may also thereby be described in terms of any combination of individual members or subgroups of members of Markush groups. Additionally, it should be noted that embodiments and features described in the context of one of the aspects and/or embodiments of the present invention may also apply mutatis mutandis to all the other aspects and/or embodiments of the invention. For instance, the thermoplastic polymers and the various types of pre-treatments of cellulose-containing fibers described in connection with the processes for producing granules naturally also apply mutatis mutandis in the context of the processes for producing biocomposites of the invention, all in accordance with the present invention as such.

As will be apparent from the description and the examples, the term “granules” relates to small particles produced by the process of the present invention, with sizes ranging from 4 μm to 1 cm. The term “cellulose-containing fibers” pertains to fibrous material obtained from suitable sources of material comprising cellulose-containing fibers, e.g. chemical pulp and/or thermo-mechanical pulp, chemo-thermomechanical pulp, and/or any other material comprising cellulose. The terms “disintegration environment”, “disintegration medium”, “thermoplastic polymer” and “thermoplastic matrix” are used substantially interchangeably and relate to the medium in which disintegration of cellulose-containing fibers is to take place. The terms “microfibrils”, “nanofibrils”, “nanofibers”, and “nanofibrillated fibers” “microfibrillated cellulose” relate to fibers obtained from disintegration of cellulose-containing fibers, with lengths ranging from 1 to 200 μm and widths ranging from 5 to 1000 nm. The terms “downstream disintegration” and “downstream processing” refer to processing of said granules in which the modified cellulose fibres are fully or partly disintegrated into cellulosic nanofibers.

Firstly, the present invention relates to a process for preparing granules comprising cellulose-containing fibers, wherein the process comprises the steps of: (a) pre-treating the cellulose-containing fibers, in order to introduce charges and/or to swell the cell wall, to facilitate downstream disintegration (b) adding at least one type of thermoplastic polymer to the pre-treated cellulose-containing fibers, and, (c) finally, compounding the obtained mixture, in order to obtain said granules. Eventually, the cellulose-containing fibers are intended to disintegrate into smaller, stronger units approaching microfibrillated cellulose in character, depending on the degree of disintegration

Washing and filtration steps may optionally be included, for instance in order to remove pre-treatment agents and excess solvent. Further, an optional homogenization step (b′) can be carried out between steps (b) and (c), to facilitate further processing of the mixture.

Step (c) may be carried out using a number of techniques/equipment known to a person skilled in the art, such as for instance extrusion, using either single or multiple screw(s), injection moulding, rotating blade machine, batch mixing, homogenization, and/or compression moulding.

The thermoplastic polymers utilized in the present invention may be selected from the group comprising, but not limited to, amylopectin, amylose, pure potato amylopectin, polylactic acid, polyhydroxy butyrate, polypropylene, polyethylene, polyvinylchloride, polyesters, polyamides, thermoplastic elastomers, natural rubber, synthetic rubbers meant for vulcanization, injection mouldable thermoset compounds and polycaprolactone, and/or any combinations thereof. Additionally, various combinations of thermoplastic polymers may be utilized in the present invention, as certain types of combinations potentially endow altered, desired characteristics to the disintegration medium.

The process of the present invention may comprise adding at least one non-volatile plasticizer, in order to facilitate production of the cellulose-containing fiber granules and to optimize the properties of the resulting nanofibrils. Non-volatile plasticizers may be selected from the group that comprises, but that is not limited to, low molecular mass carbohydrates, sucrose, glucose, fructose, polyethylene glycol, urea, glycerol, sorbitol, and/or amide-based plasticizers, and/or any combinations thereof. Adding a volatile plasticizer, such as for instance water, to the disintegration medium, either together with the non-volatile plasticizer or alone, may be an alternative if the water already present is not sufficient. The addition of plasticizer(s) may be more relevant for certain types of polymers, such as for instance starch, but including plasticizer(s) may be beneficial for other types of thermoplastic polymers as well. Furthermore, in order to facilitate the processing, a processing aid, such as for instance stearic acid, gluten, and/or magnesium stearate, and/or any combinations thereof, may be added to the mixture of thermoplastic polymer and cellulose-containing fibers. The processing aid may be present in a concentration ranging from 0-15%, preferably O-5% (w/w), preferably stearic acid at 0-5% (w/w). The ratio of thermoplastic polymer to non-volatile solvent may be between 10 to 6 and 10 to 1, respectively. The ratio of thermoplastic polymer to non-volatile solvent may preferably be 10 to 3, respectively. Analogously, if water is added as a volatile plasticizer, similar ratios are relevant. Other common additives used in plastics for inter alia UV-protection, stabilization, and/or pigmenting may be part of the material composition.

The pre-treatment of the cellulose-containing fibers of the present invention may be carried out using any one of a number of approaches, such as for instance oxidation, hydrolysis, enzymatic pretreatment and/or addition of suitable compounds swelling the cell wall, such as for instance polyethyleneglycol (PEG). Oxidation methods may be selected from the group comprising, but not limited to, TEMPO/NaClO/NaBr oxidation, carboxymethylation, and/or the cationic modification method. TEMPO/NaClO/NaBr oxidation, for instance, is carried out through suspending cellulose-containing fibers in water, adding TEMPO and sodium bromide, and, finally, adding sodium hypochlorite in a dropwise manner, in order to start the reaction. The decrease in pH caused by the formation of glucoronic acid is counteracted with the addition of sodium hydroxide, in order to maintain pH at approximately pH 10. As a chemical pre-treatment of cellulose-containing fibers, oxidation is highly advantageous as it only requires rather mild conditions and as it results in fiber swelling, which may be exploited for the downstream processing steps. Nevertheless, other methods for swelling the cell wall and/or introducing charges, known to a person skilled in the art, may, as abovementioned, also be utilized.

The final concentration of the fibers in the mixture of step (c) may be anywhere in the range between 0.1 to 70%, preferably 10-70%, more preferably 20-70%, even more preferably 30-70%, and preferably as high as possible. Obtaining granules with a high cellulose-containing fiber content is inherently difficult, but as a high fiber content correlates with improved composite properties, it is highly desirable to attain granules with a maximized amount of fibers. One of the major advantages of the present approach is that higher nanocellulose contents are possible since cellulose-containing fiber disintegration takes place during the processing, not before. Granules containing disintegrated MFC may be used as masterbatch added to neat thermoplastics, thermosets or rubbers for further processing.

The cellulose-containing fibers comprised in the granules may be substantially non-disintegrated, or may be disintegrated only to a minor extent. Granules comprising substantially non-disintegrated fibers have numerous advantages pertaining to aspects such as for instance high controllability in subsequent production for generating composites comprising individualized nanofibrils, and improved handling properties. Furthermore, the fact that the disintegration of the fibers is taking place during the processing step implies that the melt viscosity does not increase until the very last step, enabling the use of material that is otherwise difficult, if not impossible, to process with conventional techniques. However, granules comprising disintegrated cellulose-containing fibers are also within the scope of the present invention.

The present invention further concerns granules produced by any of the above processes.

A process for preparing biocomposites comprising microfibrillated cellulose and at least one thermoplastic polymer is further in accordance with the present invention. The method may comprise the steps of pre-treating cellulose-containing fibers to facilitate downstream disintegration into microfibrillated cellulose, adding at least one thermoplastic polymer to the pre-treated cellulose-containing fibers, compounding the mixture comprising thermoplastic polymer and pre-treated cellulose-containing fibers to create granules, and, finally, processing the granules of step into biocomposites. A process comprising the step of creating granules may potentially imply advantages associated with storage and shipping of granules as an intermediate product for subsequent production at another site or by another entity, optimized process flows, and sales considerations.

However, yet another process for preparing biocomposites comprising microfibrillated cellulose and at least one thermoplastic polymer in line with the present invention, comprises the steps of pre-treating cellulose-containing fibers to facilitate downstream disintegration into microfibrillated cellulose, adding at least one thermoplastic polymer to the pre-treated cellulose-containing fibers, and processing the mixture comprising at least one thermoplastic polymer and pre-treated cellulose-containing fibers into biocomposites. This direct processing of the mixture comprising at least one thermoplastic polymer and pre-treated cellulose-containing fibers may potentially involve advantages associated with inter alia a more streamlined process flow.

The pre-treatment of cellulose-containing fibers for the above processes may be selected from a group comprising at least one of oxidation, hydrolysis, enzymatic treatment, and/or addition of compounds suitable to swell the cell wall and/or to introduce charges.

The compounding step as per the process for producing biocomposite in accordance with the present invention mat be carried out using at least one technique selected from the group comprising extrusion, injection moulding, mixing, homogenization, cutting, grinding, and compression moulding. Analogously, the step of processing the mixture comprising at least one thermoplastic polymer and pre-treated cellulose-containing fibers into biocomposites may be carried out using at least one technique selected from the group comprising extrusion, hot-pressing, injection moulding, mixing, homogenization, and compression moulding.

The present invention also pertains to biocomposites produced by any of the processes in accordance with the invention.

The present invention further pertains to granules comprising between approximately 5 and 70% pre-treated cellulose-containing fibers and between approximately 30 and 95% thermoplastic polymer. Additionally, depending on factors such as type of thermoplastic polymer and cellulose-containing fiber content, the granules may comprise plasticizers and processing aids and other additives. For instance, at least one non-volatile plasticizer may be included, at a concentration ranging from approximately 5% to 35%. Further, at least one volatile plasticizer may be included in the granules, with a concentration ranging from approximately 5% to 35%, and furthermore optionally between approximately 0 and 5% of a processing aid.

The thermoplastic polymers utilized in the present invention may be selected from the group comprising, but that is not limited to, amylopectin, amylose, pure potato amylopectin, polylactic acid, polyhydroxy butyrate, polypropylene, polyethylene, polyvinylchloride, polyesters, and polycaprolactone, and/or any combinations thereof and thermoplastic elastomers, rubbers for vulcanization and thermosets for injection or compression molding. Further, various combinations of thermoplastic polymers may be utilized in the present invention, as certain types of combinations potentially endow altered, desired characteristics to the disintegration medium. Additionally, various combinations of thermoplastic polymers may be utilized in the present invention, as certain types of combinations potentially endow altered, desired characteristics to the granules.

The granules may, as abovementioned, additionally comprise at least one plasticizer, in order to optimize the properties of the resulting nanofibrils and to facilitate processing. Non-volatile plasticizers may be selected from the group that comprises, but that is not limited to, low molecular mass carbohydrates, sucrose, glucose, fructose, polyethylene glycol, urea, glycerol, sorbitol, and/or amide-based plasticizers, and/or any combinations thereof. Volatile plasticizer, such as for instance water or other types of aqueous solutions, for facilitated granule plasticizing as well as for improved properties of the granules, may also be included, either alone or in combination with the non-volatile plasticizers. The volatile plasticizer may be added separately to form part of the granules, or it may already be present as a solvent for the cellulose-containing fibers. Furthermore, in order to facilitate obtaining the granules and to improve further downstream processing, a processing aid, such as for instance stearic acid, gluten, and/or magnesium stearate, and/or any combinations thereof, may form part of the granules.

The processing aid may be present in a concentration ranging from 0-15%, preferably 0-5% (w/w), preferably stearic acid 0-5% (w/w). The ratio of thermoplastic polymer to non-volatile solvent may be between 10 to 6 and 10 to 1, respectively. The ratio of thermoplastic polymer to non-volatile solvent may preferably be 10 to 3, respectively. Similar ratios are relevant for volatile plasticizers as well.

The cellulose-containing fibers comprised in the granules may be pre-treated for instance through oxidation, using anyone of a number of oxidation methods, selected from the group comprising, but not limited to, TEMPO/NaClO/NaBr oxidation, carboxymethylation, ionic solvent oxidation, and/or the cationic modification method. Further pre-treatment approaches, known to a person skilled in the art, such as hydrolysis and addition of suitable modifying agents, such as PEG, are also within the scope of the present invention.

The present invention additionally relates to downstream uses of the above granules for various purposes, such as for instance in a method for producing composites comprising disintegrated fibers, using a method selected from the group comprising single or multiple screw extrusion and/or injection moulding. Further uses as per the present invention pertain to the production of composite materials using commonly known techniques, but other uses for said granules known to a person skilled in the art are naturally within the scope of the present invention.

### EXAMPLES

### Materials and Methods

Pure potato amylopectin was provided by Lyckeby AB (Kristianstad, Sweden). Glycerol with a purity of approximately 87% was supplied by Merck (Darmstadt, Germany). Stearic acid was supplied by Sigma Aldrich. Never-dried bleached sulphite pulp was provided by Nordic Paper Seffle AB (Sweden). Mixing of the material was carried out in a Brabender Plasticorder (Duisburg, Germany). Extrusion of the granules was carried out using a single screw extruder from AXON AB Plastic Machinery (Nyvang, Sweden). The mechanical properties of the extrudates were determined in tension using a Minimaterial Tester 2000 equipped with a load cell of 200N. The dynamic mechanical analysis tests were performed on a DMA Q800 equipment from TA Instruments. The thermogravimetric analysis and the differential scanning calorimetry was performed on a METTLER TOLEDO Instrument. A Hitachi S-4800 scanning electron microscope was utilized for visualization of the extruded granules.

### Modification of Cellulose Containing Fibres

### Example 1

### TEMPO-Oxidation of Fibers

Never dried, beaten wood pulp (dry weight 10 g) was dispersed in 750 mL H₂O. 2,2,6,6-Tetramethylpiperidine 1-oxyl (TEMPO) (0.125 g) and sodium bromide (1.25 g) were dissolved in 50 mL deionized water, and thereafter added to the cellulose fiber suspension. A 10 wt % sodium hypochlorite (NaOCl) solution (50 mmol) was adjusted to pH 10 with 1 M hydrochloric acid (HCl). The TEMPO mediated oxidation was started by addition of the NaOCl solution to the slurry, and the reaction was allowed to proceed at RT and pH 10, adjusted by addition of sodium hydroxide (NaOH) solution (0.5 M). When no adjustment of pH with NaOH was required the reaction is quenched with 15 mL ethanol, and then neutralized to pH˜7-8 with HCl-solution. Thereafter, the fibres were thoroughly washed via filtration.

### Example 2

### Carboxymethylation of Fibers

Solvent exchange never dried, beaten wood pulp (dry weight 10 g) to ethanol via repeatedly washing and filtration in 100 mL ethanol. Soak the solvent exchanged fibres in a solution of monochloricacetic acid/isopropanol (1 g/50 mL) for 30 min. Transfer the fibres into a heated solution of sodium hydroxide (NaOH) (1.6 g), methanol (50 mL), and isopropanol (200 mL). Allow the reaction to proceed for 60 min before washing the fibres by dispersion and filtration in deionized water, acetic acid solution (0.1M), and deionized water. Finally, soak the carboxymethylated fibres in sodium bicarbonate solution (4 wt %) for 60 min and then thoroughly wash them with deionized water.

### Example 3

### Enzymatic Pre-Treatment of Fibers

Disperse never dried refined softwood pulp (100 g) in phosphate buffer (250 ml, prepared from solutions of KH₂PO₄ and Na₂HPO₄ so that pH was 7±0.2). Add endoglucanases (1.7 μL) and incubate at 50° C. for 2 h, during this time mix the sample manually every 30 min. Wash the pulp with deionized water and then denaturate the endoglucanases at 80° for 30 min. Wash again with deionized water before refining the fibers in a refiner to obtain the pre-treated fibers.

a. Sample Preparation

Starch, glycerol, and water were mixed at various ratios between 10:6:6 and 10:1:1, notably 10:3:3, to prepare the disintegration environment. Stearic acid was included in the mixture at various concentrations ranging from 0-15%. Cellulose-containing fibers were oxidized in aqueous solution using various oxidation methods, and the percentage of oxidized fibers added to the disintegration environment was between 0-70% (cellulose-containing fibers/starch weight), predominantly 20%.

The mixing of the oxidized fibers with the disintegration medium was performed in a Brabender Plasticorder, with three differentially heated zones (120° C., 105° C., and 90° C., respectively). The obtained mixture was optionally stored over night in order to make it less sticky and easier to handle, before it was transferred to a rotating blade machine for conversion into granules. The rotating blade machine essentially comprises a feed zone where the material is added, a number of rotating blades, a removable grid with constant hole diameter, and a removable container for collecting the granules.

b. Extrusion of the Granules

The extrusion of the granules was performed using a single screw extruder with 5 heated zones and with a screw speed ranging from 20 rpm to 100 rpm. The temperature from the feed zone to the die were set at 140° C., 140° C., 150° C., 130° C., and 95° C.

c. Characterization—Mechanical properties

Immediately after the extrusion process, the extrudates were stored in a conditioned room with a controlled relative humidity (RH) and temperature of 50% and 23° C., respectively, for at least 10 days.

Concerning the part where the conditioning environment was changed, the samples were cut then stored in a desiccator with salt at 5% RH and 21° C. for at least 10 days. The ambient relative humidity and temperature were measured by a special device present in the desiccator. The samples were individually stored in zipped bags prior to testing and only opened when the tensile test was ready to be performed.

The mechanical properties of the samples were determined in tension using a Minimaterial Tester 2000 equipped with a load cell of 200N. The extrudates were of standard shape. The cross-head speed was 5 mm/min for all properties.

The Young's modulus, the tensile strength, the elongation at break and also the work-to-fracture were calculated using the Minimat software. The results were averaged over 5 to 8 samples.

d. Characterization—Density measurements

The density was calculated for each specimen extruded at 40 rpm but only on 6 specimens extruded at 80 rpm. The reason for that was to detect any differences in densities for the extrudates processed at higher screw speed.

The volume of each sample was obtained by measuring the dimensions using a digital calliper and a thickness meter. Three measurements were made of each dimension. The density was obtained by dividing the mass of the samples by its volume.

e. Characterization—Dynamic Mechanical Analysis (DMA)

The DMA tests were performed on a DMA Q800 equipment from TA Instruments. The samples were conditioned in the DMA chamber at 105° C. for 15 min prior testing. The samples were tested in tension from 25° C. to 300° C. with temperature ramp of 3° C./min.

The dimension of all samples were approximately 20×5×[0.3-0.7] mm³. The tests have been done twice to ensure the obtained results.

f. Characterization—Thermogravimetric Analysis (TGA)

The TGA was performed on a METTLER TOLEDO Instrument. The tests were carried out over a temperature range of 25° C. to 700° C. with a ramp of 10° C./min under an inert atmosphere of N2 of 50 ml/min.

g. Characterization—Differential Scanning calorimetry (DSC)

The DSC was performed on a METTLER TOLEDO Instrument. The tests were carried out over a temperature range of −50° C. to 300° C. with a ramp of 10° C./min under an inert atmosphere of N2 of 60 ml/min.

h. Characterization—Scanning Electron Microscopy (SEM)

A Hitachi S-4800 scanning electron microscope, operated at 0.5 kV, was used to capture secondary electron images of fracture surfaces. The samples were mounted in a metal holder using carbon tape and coated with a 3-4 nm layer of gold.

### Results and Discussion

Initially, the starch:glycerol:water ratio 100:50:50 (w/w/w) was chosen to produce a thermoplastic matrix. As a result of certain difficulties in the processing of the 100:50:50 samples, especially feeding of the granules in the extruder and the resulting aspect of the material (very rubbery and sticky even with 5 wt % of processing aid), another ratio had to be found.

Too low amounts of glycerol are known to result in processing difficulties and excessive glycerol may lead to glycerol exudation.

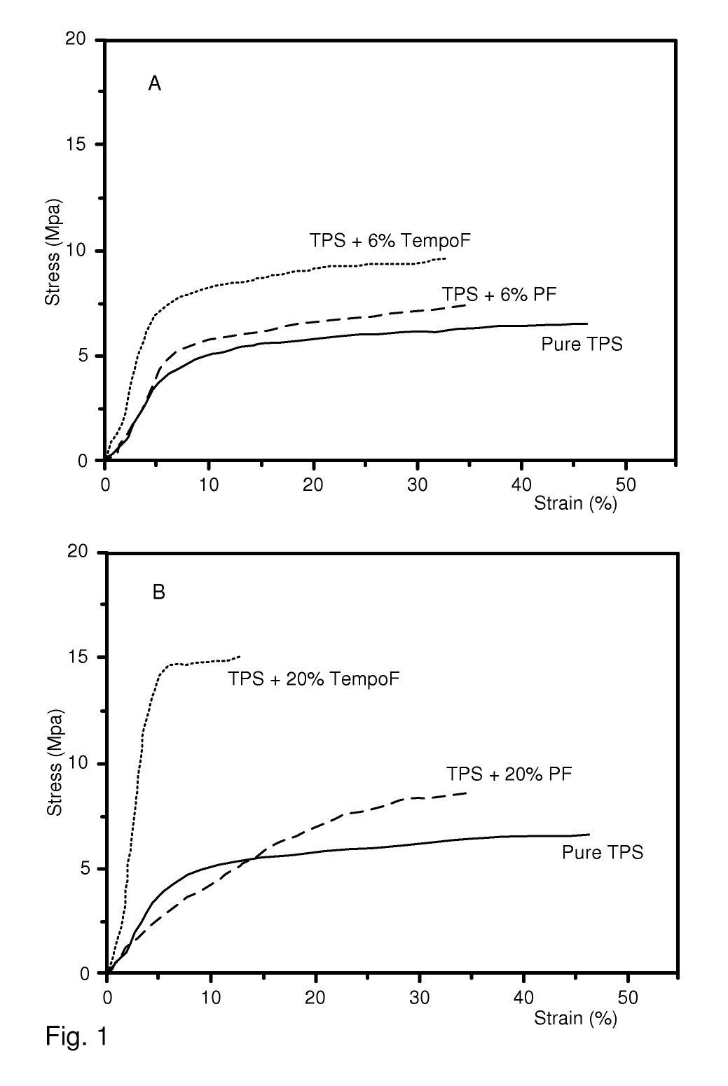

The original screw speed was 40 rpm for all trials. The Stress-Strain curves of samples containing 0, 6 and 20 wt % of fibers (treated or untreated) are represented in FIG. 1. A recap of mechanical properties and densities of all specimens produced at 40 rpm as screw speed is shown in Table 1. As can be seen from the curves, the samples containing treated fibers have better properties than samples with unmodified cellulose-containing fibers. The differences between the mechanical behaviour of the different composites are more accentuated with the addition of 20 wt % of treated fibers compared to cellulose-containing fibers. The stress-strain curves also show that the material containing treated fibers become more brittle with the addition of fibers. In contrast, the addition of untreated cellulose-containing fibers in the matrix shows only small changes in elongation at break and E modulus (decrease). As can be seen in Table 1, the elastic modulus of the composite TPS+20% TempoF is almost 9 times higher than the composite TPS+20% PF elastic modulus, which are 489 MPa and 55 MPa respectively. The tensile strength is almost the double for the composite TPS+20% TempoF compared to the one with untreated fibers. The tensile strengths are 8.36 MPa and 14.88 MPa for the TPS+20% PF and TPS+20% TempoF respectively.

TABLE 1
Densities and mechanical properties of composites made with TPS and
cellulose-containing fibers (treated or untreated) and extruded at
40 rpm. At least 10 days at 50% RH and 23° C. for conditioning.

| | | YOUNG'S | TENSILE | ELONGATION | WORK-TO- |
|---|---|---|---|---|---|
| | DENSITY | MODULUS | STRENGTH | AT BREAK | FRACTURE |
| SAMPLE | (kg/m) | (MPa) | (MPa) | (%) | (MJ/m) |
| Neat TPS | 1330 | 99.35 ± 1.37 | 6.40 ± 0.45 | 47.46 ± 4.11 | 2.58 ± 0.39 |
| TPS + 1% PF | 1450 | 77.26 ± 5.50 | 6.01 ± 0.16 | 41.99 ± 3.60 | 2.22 ± 0.30 |
| TPS + 3% PF | 1474 | 87.97 ± 6.50 | 6.26 ± 6.29 | 34.58 ± 4.90 | 1.82 ± 0.37 |
| TPS + 6% PF | 1450 | 100.76 ± 16.73 | 7.29 ± 0.18 | 33.98 ± 2.90 | 2.08 ± 0.21 |
| TPS + 20% PF | 1453 | 54.71 ± 6.83 | 8.36 ± 2.60 | 47.56 ± 15.60 | 2.84 ± 0.60 |
| TPS + 1% TempoF | 1402 | 106.89 ± 6.37 | 7.15 ± 0.14 | 48.08 ± 3.86 | 3.14 ± 0.24 |
| TPS + 3% TempoF | 1403 | 198.26 ± 14.91 | 8.10 ± 0.25 | 21.65 ± 2.77 | 1.60 ± 0.16 |
| TPS + 6% TempoF | 1410 | 203.66 ± 35.10 | 9.31 ± 0.51 | 34.35 ± 3.69 | 3.12 ± 0.46 |
| TPS + 20% TempoF | 1451 | 489.19 ± 37.80 | 14.88 ± 0.58 | 13.65 ± 3.07 | 1.64 ± 0.41 |

The work-to-fracture of the composites has been evaluated from the area under the Stress-Strain curves. From Table 1, it can be seen that the values of the work-to-fracture do not appear to follow any trend. However, the highest values obtained for the work-to-fracture are 3.14 MJ/m³ and 3.12 MJ/m³ for the composites TPS+1% TempoF and TPS+6% TempoF, respectively. A lower value, 2.86 MJ/m³, for the composite TPS+20% PF is also observed but this is also the highest value obtained for the composites containing cellulose-containing fibers. If we consider only these values, we can in fact see an improvement in the work-to-fracture due to the presence of treated and untreated fibers. But this cannot be considered as a true statement since the other values are totally different.

The specimens with 20 wt % of treated fibers exhibit the lowest work-to-fracture, which means that this material has the most brittle mechanical behaviour compared to the others. Besides, this brittle behaviour is also supported by the lowest elongation at break, highest Young's modulus and highest tensile strength, which are 13.65%, 14.88 MPa and 489.19 MPa respectively.

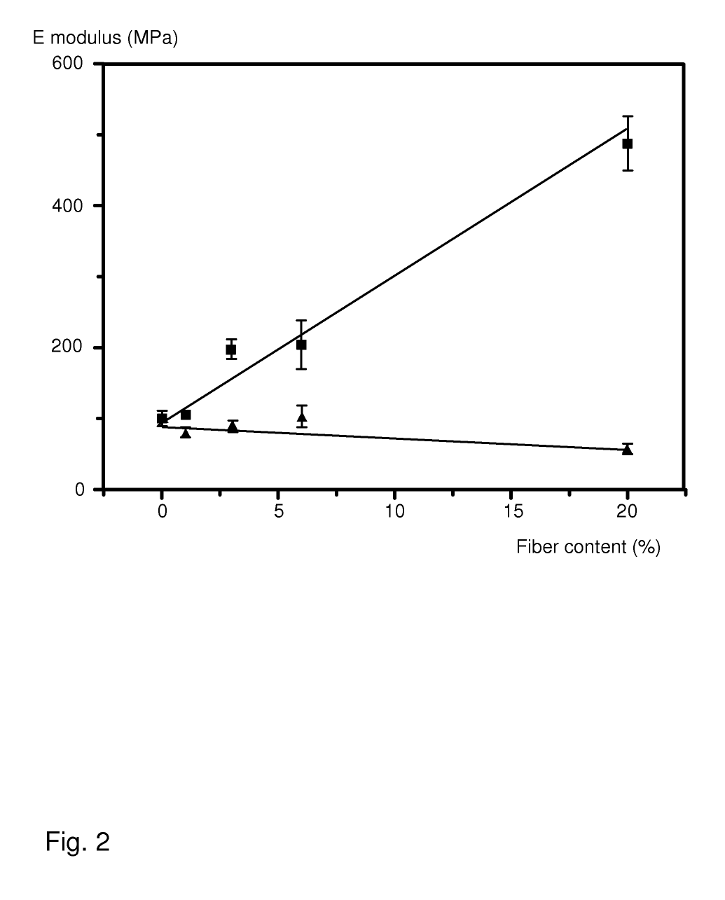

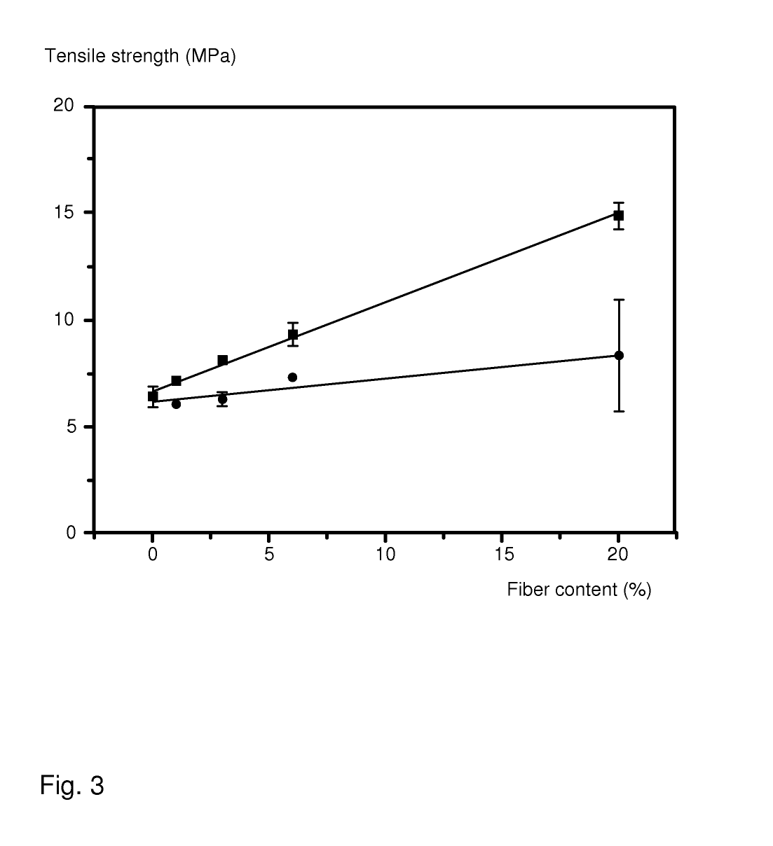

FIG. 2 and FIG. 3 represents the E modulus and the tensile strength of the composites according to the fiber content in wt % (PAP basis).

In FIG. 2, again, notable differences can be seen in evolution of the Young's modulus for the two types of composites (with treated and untreated cellulose-containing fibers). The specimens with untreated cellulose-containing fibers exhibit a small decrease in stiffness with increasing fiber content.

The composite with 20% of cellulose-containing fibers has a lower Young's modulus than the TPS matrix itself. This result was actually unexpected because the Young's modulus should have been at least better with a higher content of cellulose-containing fiber than without any filler. A big dispersion and diffusion of cracks in the TPS matrix and the remaining of the randomly oriented filler network could have lead to this drop in the elastic modulus.

However, FIG. 3 shows an increase in tensile strength for both types of composites. The increase is more significant in the case of the treated fibers. At a fiber content of 20%, for both treated and untreated cellulose-containing fibers, the composites exhibit tensile strengths of 14.88 MPa and 8.36 MPa respectively. The tensile strength of the treated fibers+TPS composite is almost 2 times higher than the one for the original cellulose-containing fibers+TPS composite. Besides, it is noticeable that the tensile strength value of the specimen with untreated cellulose-containing fibers at 20 wt % has a large standard deviation compared to the others.

The important increase of the mechanical properties of the specimens containing TEMPO-oxidized fibers compared to those of specimens containing untreated cellulose-containing fibers confirm the effect of the oxidizing treatment. The oxidation of the cellulose fibers may have contributed to their better dispersion in the TPS matrix and also their disintegration.

Further, the mechanical differences between the extruded composites at different screw speed have been explored. A screw speed of 80 rpm was chosen to evaluate if a higher screw speed would have a positive influence in the disintegration process of the treated cellulose-containing. A higher screw speed would increase the shear inside the extruder and thereby induce a better disintegration. But a higher shear would also potentially degrade the TPS matrix, as thermoplastic starch is rather sensitive and it may be submitted to hydrolysis with subsequent reduced properties.

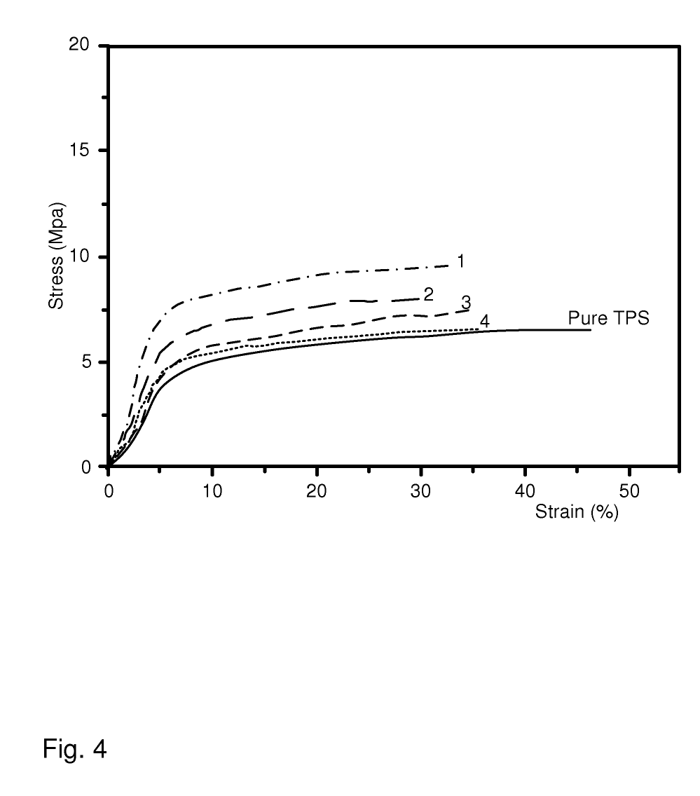

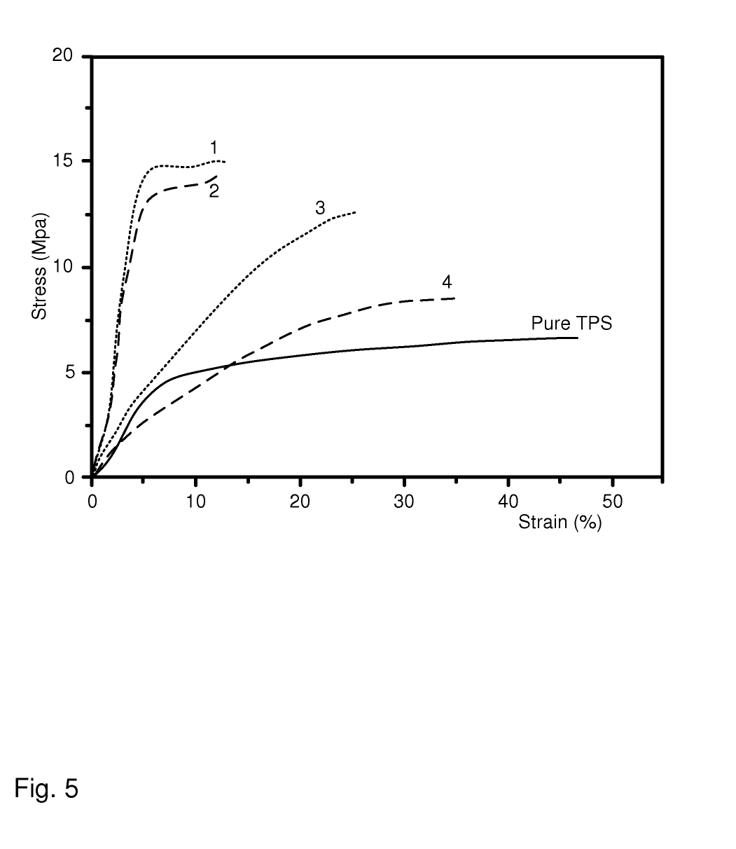

FIG. 4 and FIG. 5 show the stress-strain curves of the different composites processed at 40 and 80 rpm with 6% of fibers (both types) and 20% of fibers (both types) respectively.

At first glance, no significant improving in the mechanical properties of the composites is observed with increasing screw speed. On a contrary, there is a decrease in the mechanical properties at higher screw speed, except for the specimen with 20% of cellulose-containing fibers. As shown in Table 2, its Young's modulus and tensile strength seems actually to be improved by the higher shear during its production. The Young's modulus increases from 54.71 MPa at 40 rpm to 79.79 MPa at 80 rpm. The tensile strength increases from 8.36 at 40 rpm to 12.17 MPa at 80 rpm.

This peculiar increase in the properties of this composite compared to the global decrease in the properties could perhaps be explained by a better homogenization of the fibers in the TPS matrix. This better dispersion led to a lower content of voids which could explain this behaviour. The extrusion of the composite TPS+20% PF was extremely difficult, and the extrudate had difficulties to come out of the die. The augmentation of the shear stress along the extruder barrel may have improved this dispersion and also the viscosity induced by the high content of cellulose-containing fibers which certainly has caused the slow production of this sample.

TABLE 2
Young's moduli and tensile strengths of composites TPS + Fibers
(both types) for the two different screw speeds 40 rpm and 80 rpm.

| | YOUNG'S MODULUS | | TENSILE STRENGTH | |
|---|---|---|---|---|
| | (MPa) | | (MPa) | |
| SAMPLE | 40 rpm | 80 rpm | 40 rpm | 80 rpm |
| Neat TPS | 99.35 ± 1.37 | — | 6.40 ± 0.45 | — |
| TPS + 1% PF | 77.26 ± 5.50 | 111.50 ± 6.56 | 6.01 ± 0.16 | 6.91 ± 0.41 |
| TPS + 3% PF | 87.97 ± 6.50 | 72.42 ± 7.47 | 6.26 ± 6.29 | 6.07 ± 0.19 |
| TPS + 6% PF | 100.76 ± 16.73 | 110.45 ± 2.86 | 7.29 ± 0.18 | 6.71 ± 0.20 |
| TPS + 20% PF | 54.71 ± 6.83 | 79.79 ± 11.68 | 8.36 ± 2.60 | 12.17 ± 0.98 |
| TPS + 1% TempoF | 106.89 ± 6.37 | 48.04 ± 7.75 | 7.15 ± 0.14 | 4.98 ± 0.32 |
| TPS + 3% TempoF | 198.26 ± 14.91 | 138.55 ± 17.28 | 8.10 ± 0.25 | 7.30 ± 0.24 |
| TPS + 6% TempoF | 203.66 ± 35.10 | 160.53 ± 16.77 | 9.31 ± 0.51 | 8.10 ± 0.32 |
| TPS + 20% TempoF | 489.19 ± 37.80 | 455.33 ± 40.53 | 14.88 ± 0.58 | 13.77 ± 0.91 |

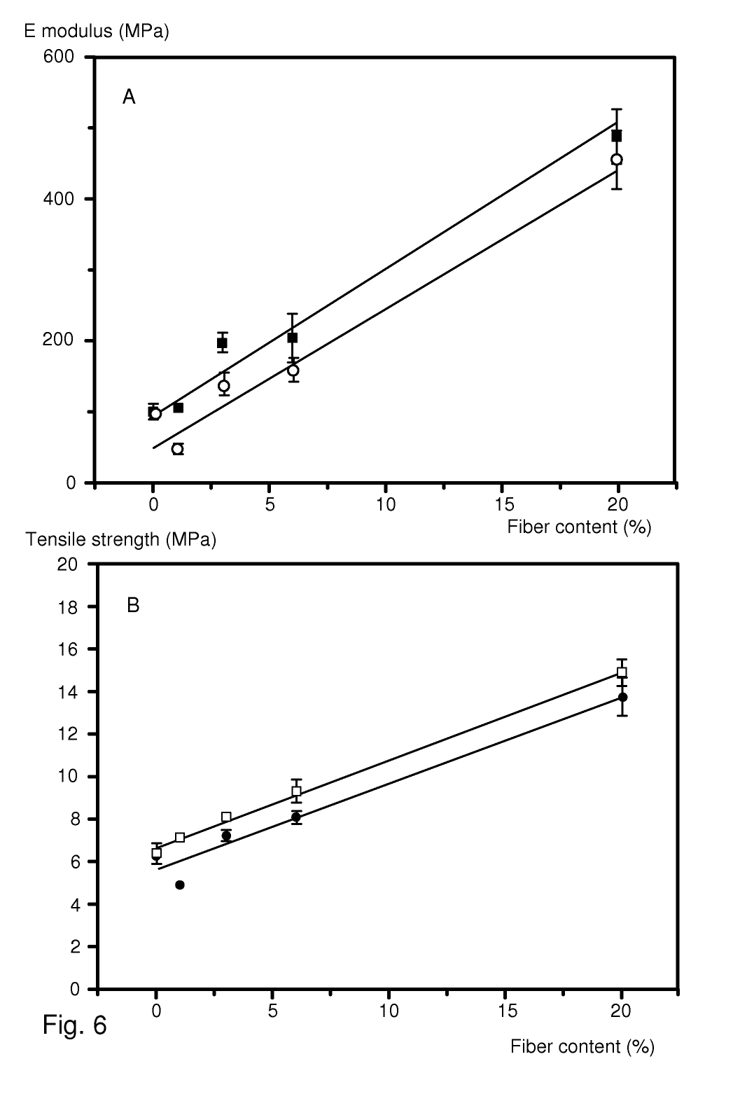

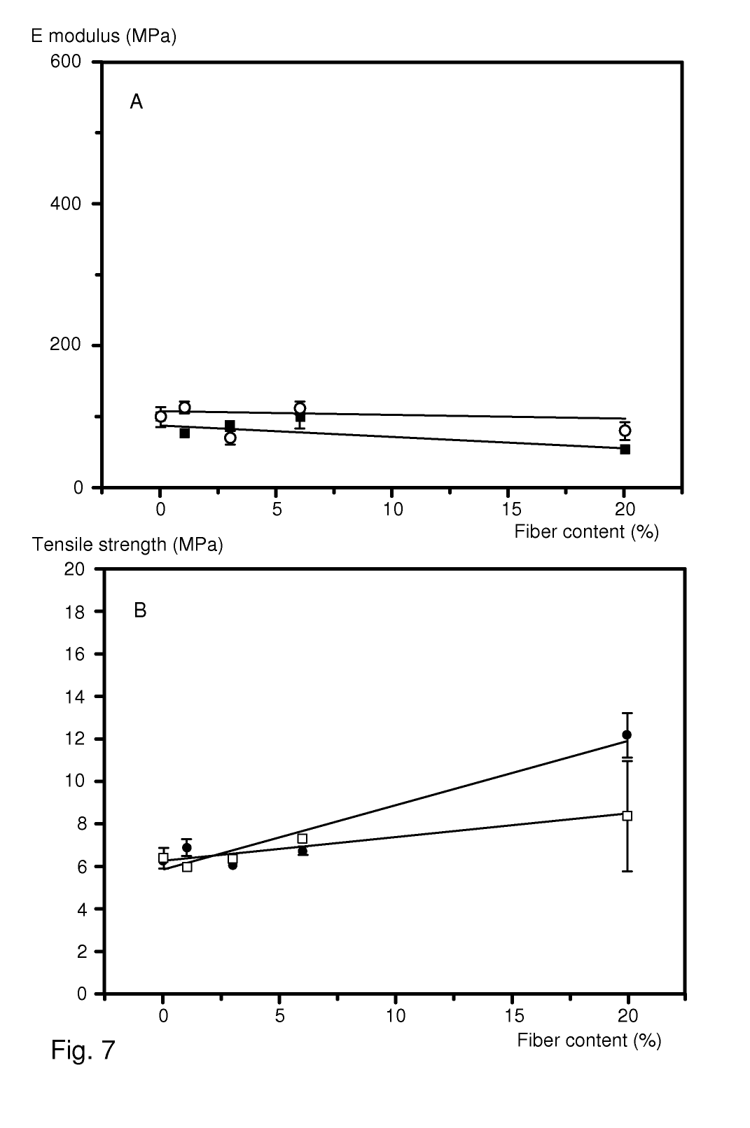

FIG. 6 and FIG. 7 show the evolution of the elastic modulus and the tensile strength versus the fiber content for the two different screw speeds 40 and 80 rpm. We can see that for the composites containing TEMPO-oxidized fibers, the augmentation of the screw speed from 40 to 80 rpm did not give the expected results. On the contrary, a higher shear gave lower mechanical properties. However, for the composites containing unmodified cellulose-containing fibers, the high shear seems to have enhanced the physical properties of the composites which led to better mechanical properties. Besides we can see that the evolution of both mechanical properties for the composites with treated fibers, even at higher shear, kept the same trend but with lower values.

As presented in Table 2, E moduli and tensile strength of the composites with treated fibers have lower values when we increase the screw speed. But the decrease of the properties is less significant when the amount of fibers in the composites is higher. On one hand, for the composite TPS+1% TempoF, the E modulus is 106.89 MPa and 48.04 MPa for 40 rpm and 80 rpm, respectively, i.e. approximately 50% of the value at 40 rpm. On the other hand, for the composite TPS+6% TempoF, the E modulus is 203.66 MPa and 160.53 MPa for 40 and 80 rpm, respectively, i.e. only 20% diminution.

The difference in behaviour at higher screw speed for the two types of composites (with treated or untreated fibers) is perhaps due to the incorporation of surface modified fibers with carboxylate groups. This incorporation may have contributed to the hydrolysis of the TPS matrix because of a facilitated moisture penetration inside the treated fibers introduced by this individualization of the nanofibrils inside the structure.

To conclude the mechanical properties part, the introduction of treated fibers in the TPS matrix had a great influence in the mechanical properties of the composites compared to the incorporation of untreated fibers. The properties were significantly improved even at low content (only 20% or less) of treated fibers compared to original cellulose-containing fibers. The improvement can be explained by a successful disintegration process of the treated fibers during the extrusion.

The TGA has been carried out on the main constituents of the composites; PAP, glycerol but also cellulose-containing fibers and TEMPO-oxidized cellulose-containing fibers. The degradation temperatures of the glycerol, PAP and cellulose-containing fibers are 246° C., 298° C., and 342° C., respectively. However, the cellulose-containing fibers seemed to exhibit a two-step degradation: the first degradation temperature corresponds to the depolymerisation of the cellulose chains, and the second degradation step started at 450° C., corresponding to the depolymerisation of the remaining char. Besides, the degradation temperature of the TEMPO-oxidized fibers seems to be lower.

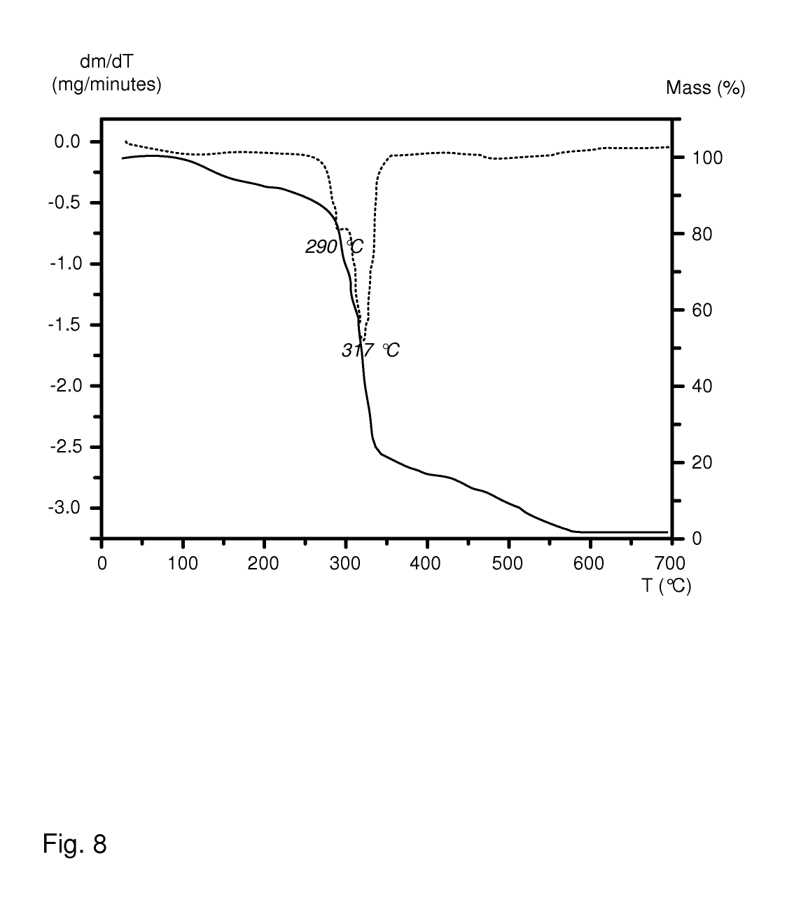

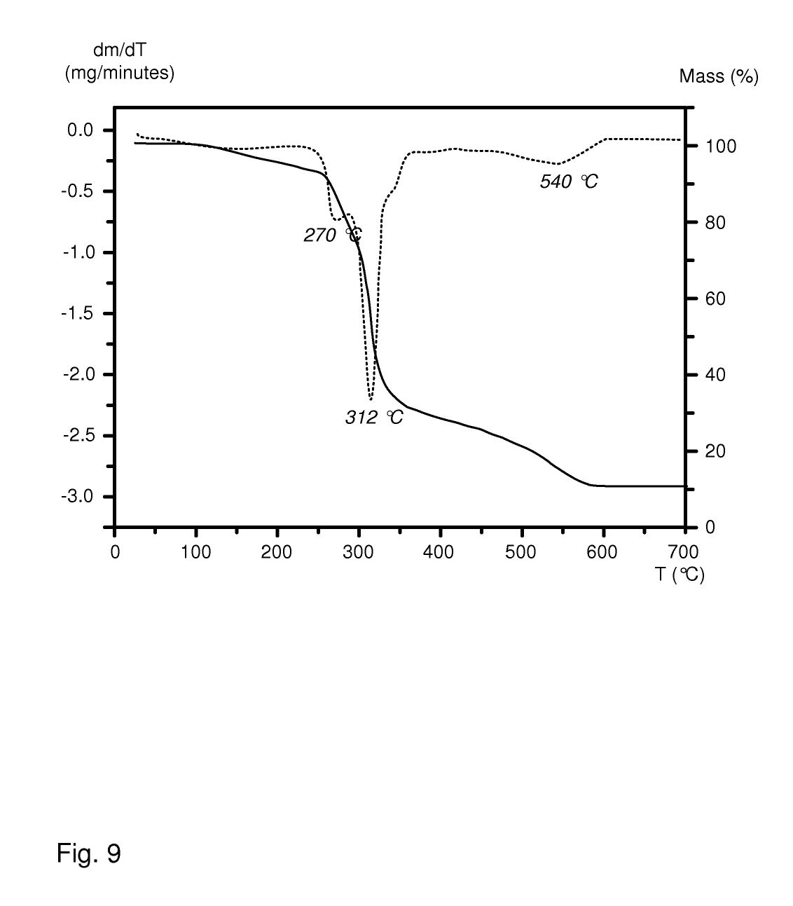

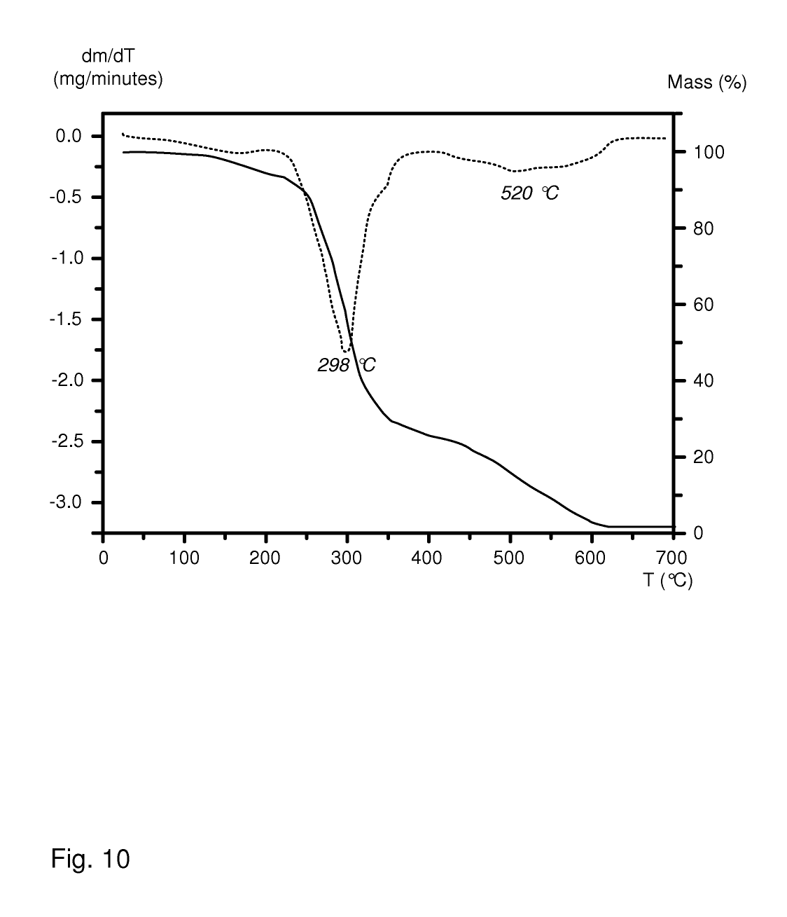

The mass evolution of the neat TPS matrix is shown in FIG. 8. The first derivative has a big peak at 317° C. and a small one at 290° C. The TGA of the composite containing 20% of cellulose-containing fibers is shown in FIG. 9. It displays the same pattern as for the neat TPS, a small peak at 270° C. and a big peak at 312° C. The composite containing 20% of treated cellulose-containing fibers starts becoming degraded at 298° C. as represented in FIG. 10.

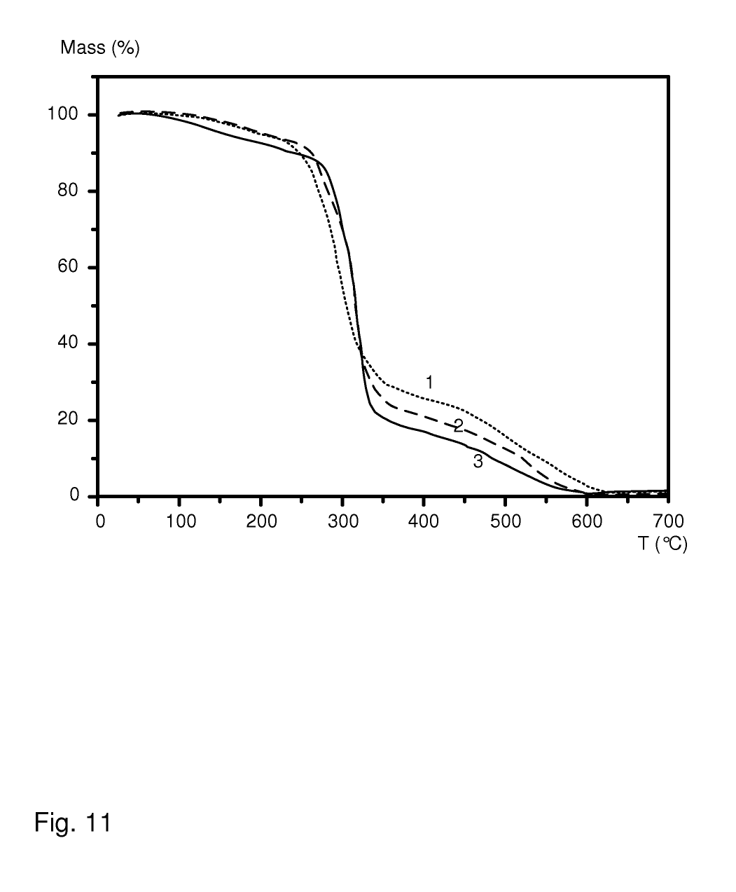

FIG. 11 shows the comparison between the TGA curves of the composites containing 20% fillers. First, we can determine that the neat matrix had a higher moisture content or also lost glycerol, since the specimen lost more weight after the evaporation of the water around 100° C.

Besides, it is noticeable, that the composites start degrading before the neat matrix. The composite containing TEMPO-oxidized fibers has the lowest degradation temperature. However, the composites seem to lose less weight than the neat matrix, which means that they are more resistant toward degradation. We see on FIG. 11 that the composite containing treated fibers lose weight less rapidly than the other composite containing untreated fibers and the matrix. This could be an implication of the presence of disintegrated fibers in the composite.

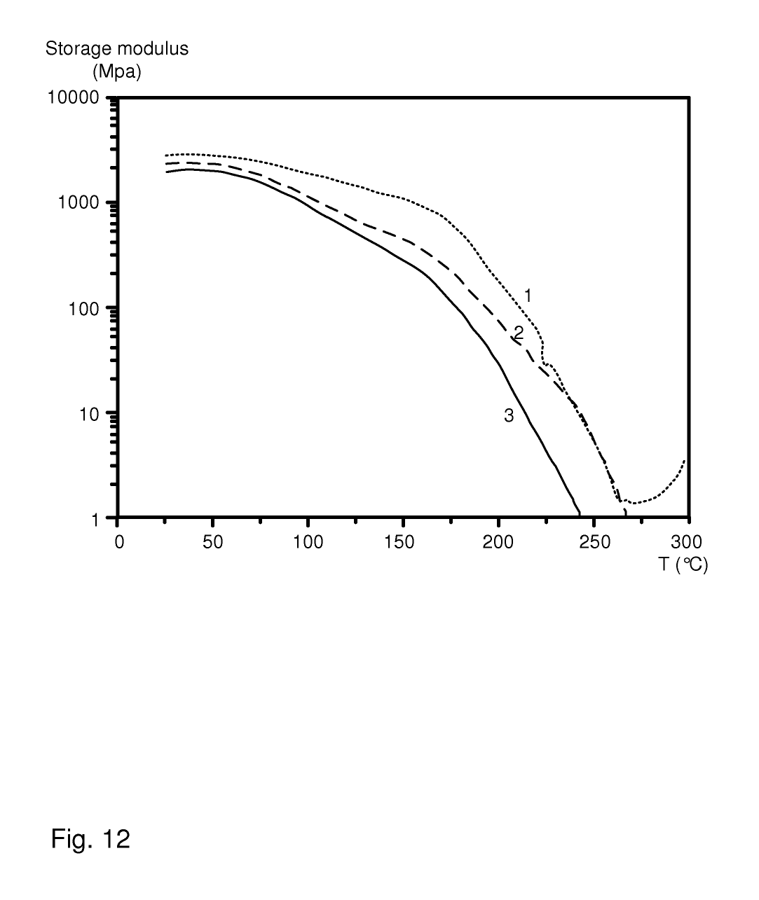

FIG. 12 shows the evolution of the storage modulus with the increasing temperature. The temperature dependence of the modulus is decreasing when fibers are added to the TPS matrix. However, the addition of treated fibers in the TPS matrix has a higher effect than the original cellulose-containing fibers. Moreover, the curves of both composites seem to have the same trend at around 225° C. The curve of the composite with treated fibers drop drastically to follow the trend of the other composite curve. This turning point could be when the TPS matrix starts to melt.

From Table 3 shows the value of the storage modulus of the neat matrix and its composites TPS+20% PF and TPS+20% TempoF at 160° C. and 280° C. At 160° C., the storage modulus of the neat matrix is 206 MPa, for the composite with 20% of untreated fibers 342 MPa, and 912 MPa for the composite containing 20% of treated fibers. The storage modulus obtained with the addition TEMPO-oxidized fibers is at least 4 times higher than the neat matrix but also almost 3 times higher than the storage modulus of the composite with untreated cellulose-containing fibers.

At 280° C., the storage modulus of the TPS matrix is 0.02 MPa, in contrast to the composite containing treated fibers which has a storage modulus of 1.4 MPa. This is actually 70 times higher than the neat matrix. Concerning the composite with untreated fibers, its storage modulus decreases from 344 MPa at 160° C. to 0.19 MPa at 280° C. Additionally, FIG. 12 clearly illustrates that the storage modulus of the composite containing treated cellulose-containing fibers stays constant after 200° C. and then increases. The storage moduli of the neat matrix and the specimen with original cellulose-containing fibers just keep decreasing with the augmentation of the temperature.

TABLE 3
Storage moduli of the neat matrix, composite with 6% of treated fibers
and the two types of composites with 20% of fibers (treated or untreated)
at two different temperatures 160° C. and 280° C.

| | Temperature | Storage Modulus |
|---|---|---|
| Sample | (° C.) | (MPa)* |
| Pure TPS | 160 | 206 |
| | 280 | 0.02 |
| TPS + 20% Pulp fibers | 160 | 344 |
| | 280 | 0.19 |
| TPS + 20% treated fibers | 160 | 912 |
| | 280 | 1.40 |
| TPS + 6% treated fibers | 160 | 391 |
| | 280 | 0.10 |

*The storage modulus values were taken directly from the DMA results with the analysis software.

Finally, the DMA gave interesting results since the composite with treated fibers seemed to have a less temperature-dependant mechanical behaviour. However, the general behaviour of a composite containing MFC is a hardly thermal dependant mechanical behaviour, which is translated into a quasi-flat evolution through the temperature increase. Thus, the DMA showed that the composite containing treated fibers had a different evolution, probably due to the presence of disintegrated fibers but also remaining “full-size” fibers.

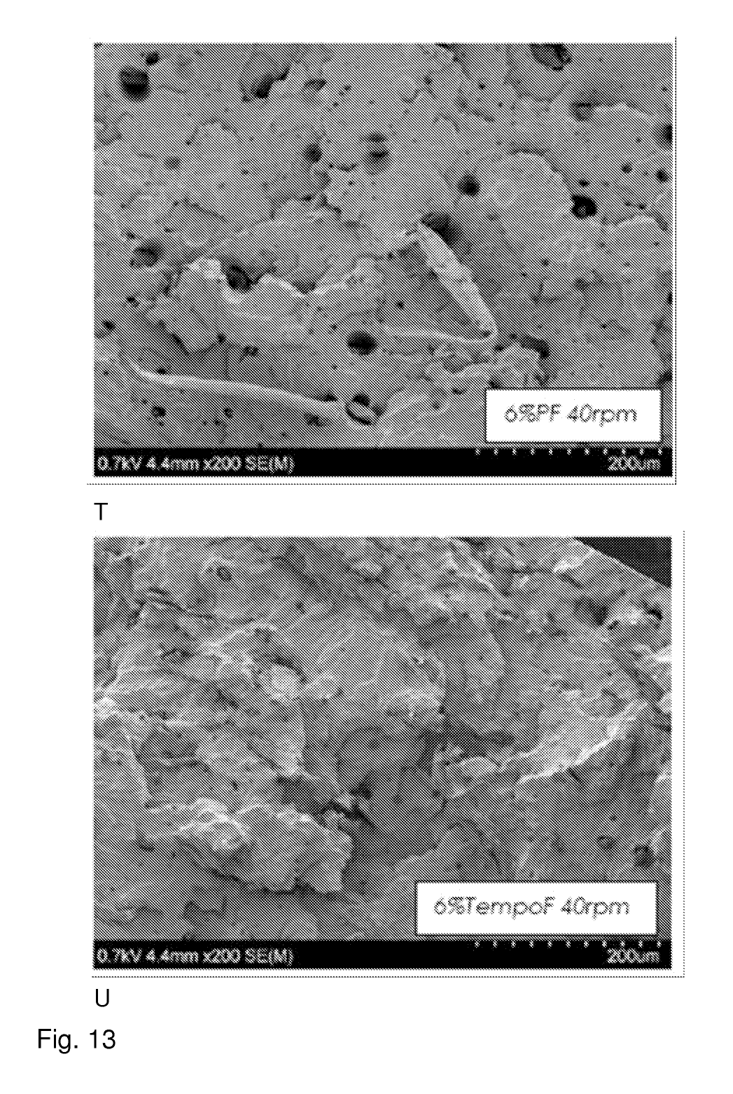

The fracture surfaces of the composites were investigated with scanning electron microscopy (SEM). FIG. 13 shows the SEM pictures of the composites with 6% untreated fibers and 6% treated fibers at the same magnification (×200). The fracture surface of the composite with 6% cellulose-containing fibers revealed numerous holes and big fibers. The holes could correspond to pull-out zones of the fibers when subjected to tensile stresses. Besides, the fracture surface of the composite with 6% treated fibers is completely different from the previous one. The fibers dispersion is finner and the general size of the present fibers seem smaller.

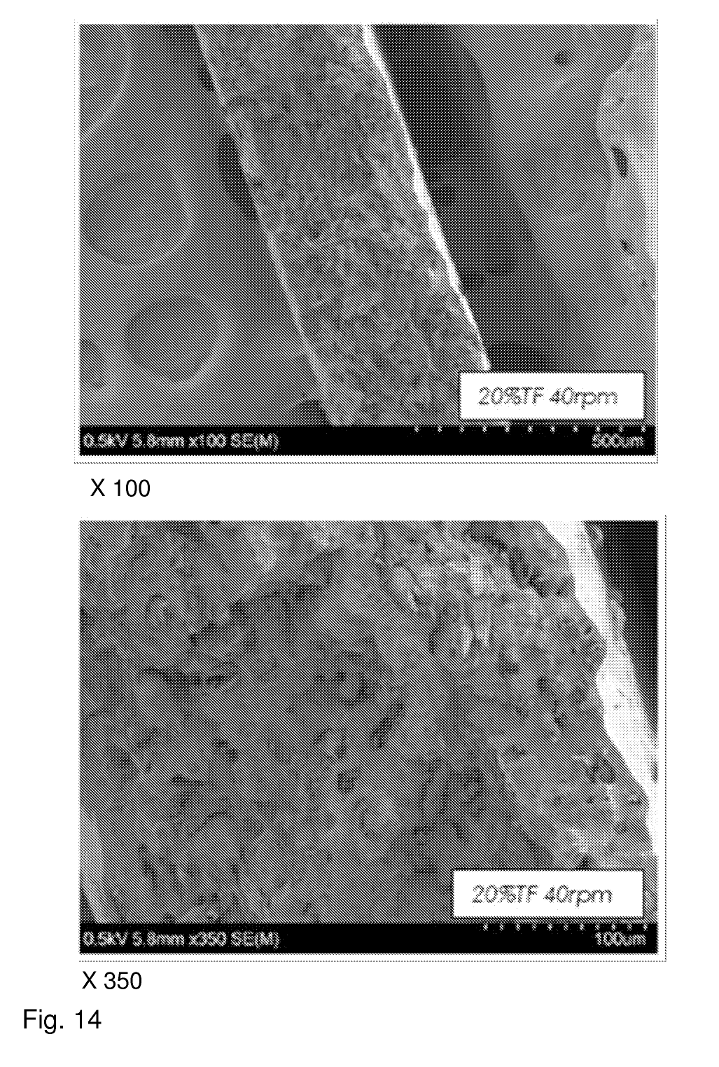

FIG. 14 represents the fracture surface of the sample containing TPS and 20% treated fibers. At lower magnification (×100), the cellulose-containing fibers can not be distinguished from the fracture surface. At higher magnification (×350), fibers with a dimension of several μm and smaller fibers can de identified, which means that a part of the treated fibers has not been disintegrated during the extrusion process.

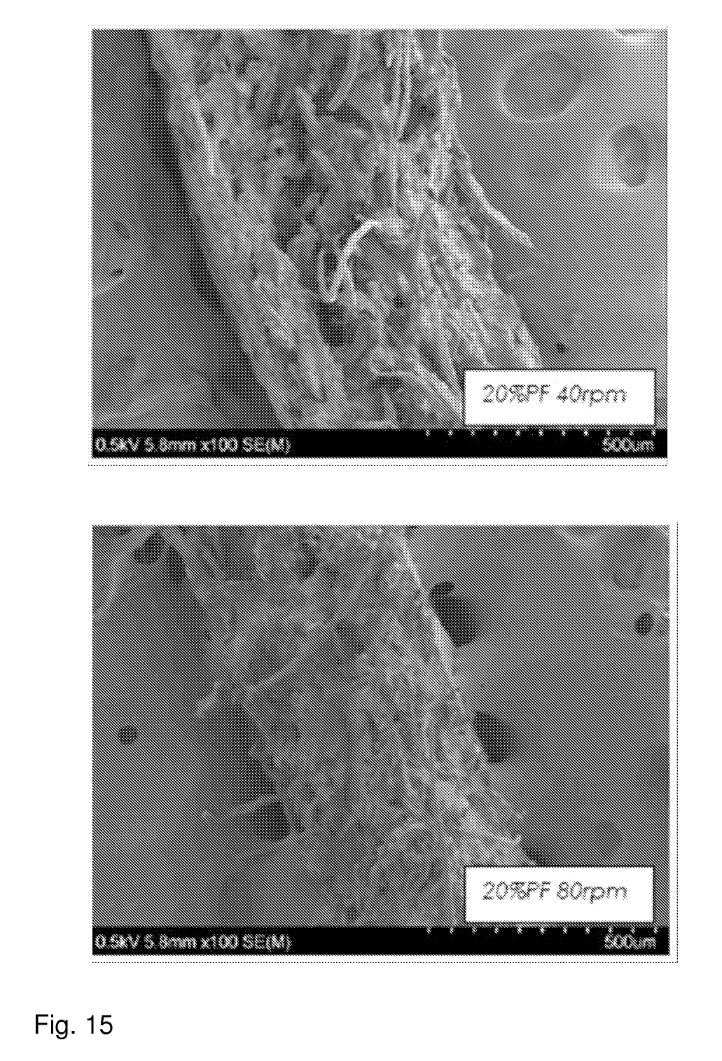

The physical differences between the samples containing 20% of untreated fibers and processed at 40 rpm or 80 rpm can be seen in FIG. 15. The sample processed at a screw rotation speed of 40 rpm exhibit voids in contrast to the sample extruded at 80 rpm. The dispersion in the specimen seems to have been improved with the highest speed procedure. This observation is directly related to the results obtained previously for the same composite. This improvement in the mechanical properties is related to the improvement of the composite structure itself.

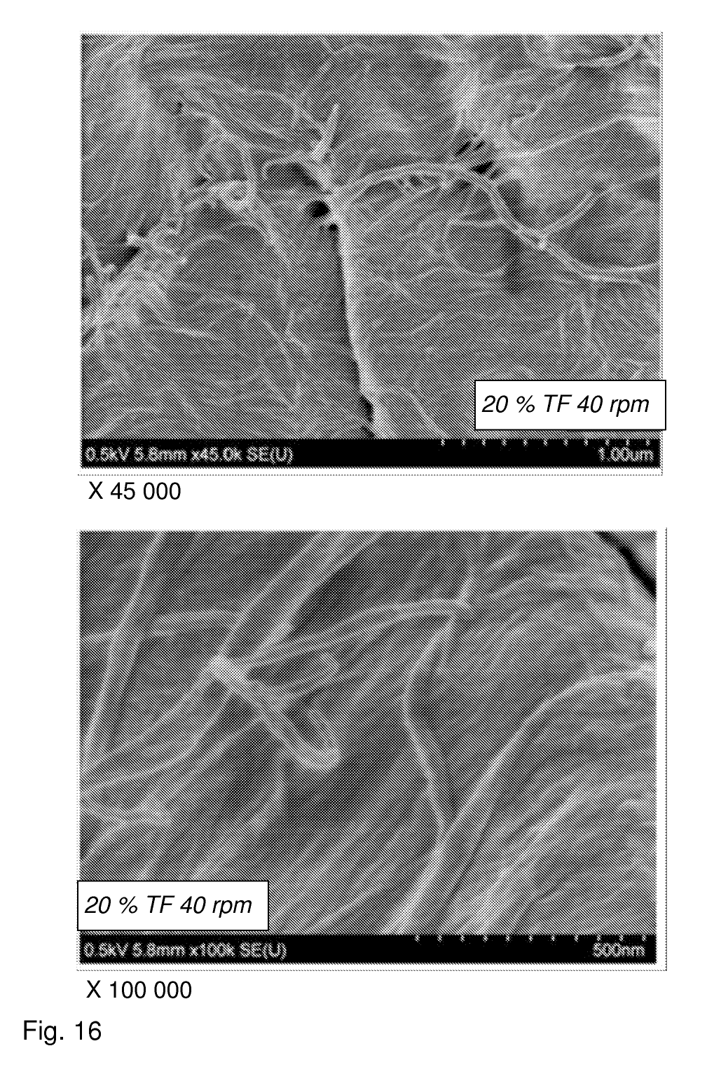

The structure of the composite containing 20% of TEMPO-oxidized fibers has been studied at higher magnification to confirm the presence of nano-sized fibers in the TPS matrix. Two of the SEM images obtained are shown on FIG. 16. We can observe the composite at a magnification of ×45000 and ×100000. The images show that the specimen definitely contains nanofibers. The detected nanofibers have a diameter in the range of 20-40 nm.

Additionally, the effect of the surrounding relative humidity on the samples was studied. To this end, a part of the samples from both types of composites (with treated and untreated fibers) were put in a desiccator with a humidity controller. The relative humidity was kept at 5% RH (±1%) and the temperature varied from 22° C. to 19° C. The samples were conditioned during at least 10 days.

The mechanical properties of the samples were tested as quickly as possible after storage. Specimens were put in separate zipped bags to avoid influence from the surrounding environment in the testing room, since the ambient relative humidity was approximately 50% RH. When the tensile test was set and ready, the specimen was taken out of its bag and tested.

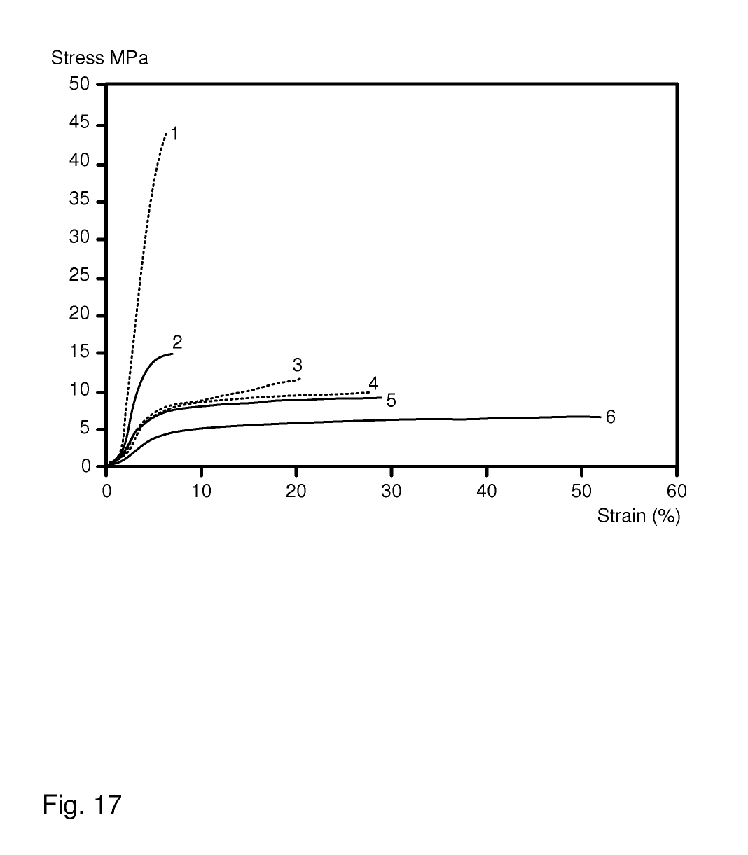

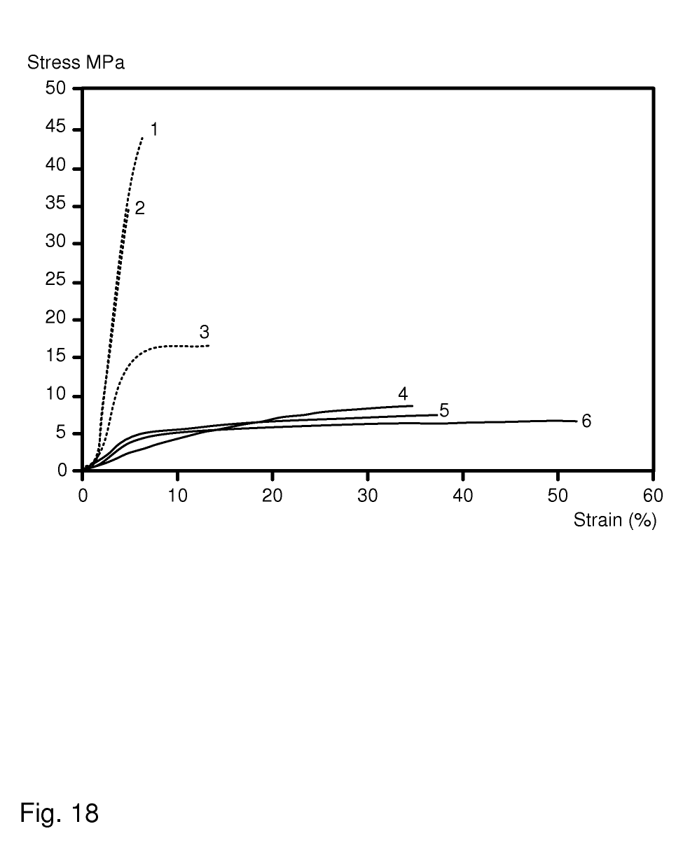

FIG. 17 and FIG. 18 show the stress-strain curves of both types of composites subjected to two different conditioning environments (5% RH and 50% RH). At first glance, it can be observed that the mechanical behaviour of both composites toward the change in relative humidity is completely different. The composites containing untreated cellulose-containing fibers seem to be more sensitive to the ambient relative humidity than the composites containing treated cellulose-containing fibers. However, their sensitivity is decreasing when increasing the fiber content. The composites with untreated cellulose-containing fibers as well as the TPS matrix, exhibit high stress at break and low elongation at break when compared to their behaviour at 50% RH.

The Young's moduli and stress at break values of the tested composites are presented in Table 4. The TPS matrix is the most sensitive to the surrounding humidity; its E modulus is coming from around 100 MPa to 1.1 GPa for 50% RH and 5% RH respectively. Its tensile strength is 7 times higher at 5% RH than 50% RH. The mechanical behaviour of the specimen TPS+6% PF is similar to the pure matrix, only specimens with 20% PF have the lowest augmentation in their mechanical properties. Concerning the composites containing treated cellulose-containing fibers the difference in behaviour seem less visible. The Young's modulus of the specimen TPS+6% treated fibers doesn't change even when put at 5% RH, the values were 203.66 MPa and 197.8 MPa at 50% RH and 5% RH respectively. Besides, the tensile strength of the latter composite was 9.31 and 9.47 MPa at 50% RH and 5% RH respectively. For the composite containing 20% of treated fibers, the conditioning at 5% RH has lowered its properties. The E modulus and stress at break were 489.19 and 14.88 MPa respectively for 50% RH. At 5% RH, the E modulus and stress at break were 225.7 and 11.50 MPa, respectively.

Both types of fibers in the TPS matrix thus had a major influence on the behaviour of their composites at 5% RH. This big change in mechanical behaviour can be explained by a phenomenon called “anti-plasticization”. This phenomenon is generally occurring in synthetic polymers when the additives are present in low amounts (generally <15%) but the mechanisms involved are not perfectly known, but the antiplasticization phenomenon appears to correspond to a decrease of the elongation and an increase of the stress.

TABLE 4
E moduli and tensile strengths of TPS and
composites conditioned at 50% RH and 5% RH.

| | E MODULUS | | STRESS AT BREAK | |
|---|---|---|---|---|
| | (MPA) | | (MPA) | |
| SAMPLE | 50% RH | 5% RH | 50% RH | 5% RH |
| TPS | 99.35 ± 13.72 | 1103.80 ± 41.49 | 6.39 ± 4.53 | 43.26 ± 2.10 |
| TPS + 6% Treated fibers | 203.66 ± 35.10 | 197.8 ± 17.56 | 9.31 ± 0.51 | 9.47 ± 0.32 |
| TPS + 20% Treated fibers | 489.19 ± 37.80 | 225.7 ± 21.18 | 14.88 ± 0.58 | 11.50 ± 0.89 |
| TPS + 6% PF | 100.76 ± 16.73 | 1021.14 ± 166.72 | 7.29 ± 1.78 | 33.34 ± 5.78 |
| TPS + 20% PF | 54.71 ± 6.83 | 449.13 ± 26.04 | 8.36 ± 2.60 | 17.19 ± 0.59 |

From the mechanical results obtained at 5% RH, we conclude that adding the fibers in the TPS matrix helped evading this anti-plasticization process. Moreover, composites with treated fibers are even less sensitive to the surrounding humidity and thus prevent the “antiplasticization” process.

The treated fibers composites may hence be less sensitive to ambient humidity due to the presence of nano-size fibers. The interaction between the individualized fibers and the disintegration medium probably leads to a reduced moisture sensitivity due to strong intermolecular interaction and constrained effects on swelling exerted by the cellulose nanofibers. Thereby, the composites containing disintegrated fibers could probably not have release all the moisture acquired during their storage at 50% RH due to a lower diffusivity of the moisture compared to a cellulose-containing fiber network. Thus, the composites with treated fibers are less sensitive to moisture which leads to no significant antiplasticization from water.

To conclude, thermoplastic matrixes and composites of such matrixes containing TEMPO-oxidized cellulose-containing fibers and untreated cellulose-containing fibers have been extruded using a screw extruder, but the processing has also been carried out using various other techniques. Composites with treated fibers showed significantly higher mechanical and thermal properties than the other specimens with untreated cellulose-containing fibers. SEM images of the fracture surfaces of the composites revealed fibers of smaller size and finer dispersion in the case of the composite with treated fibers. Both nano-scale and micron-scale fibers have been found. All the tests showed that the disintegration by extrusion of the TEMPO-oxidized cellulose in a plasticized starch matrix has occurred.

1. A process for preparing granules comprising cellulose-containing fibers, said process comprising the steps of:
(a) pre-treating cellulose-containing fibers to facilitate downstream disintegration into microfibrillated cellulose;
(b) adding at least one thermoplastic polymer to the pre-treated cellulose-containing fibers obtained in step (a);
(c) compounding the mixture obtained in step (b) to obtain said granules.

2. Process according to claim 1, wherein step (c) is carried out using at least one technique selected from the group consisting of extrusion, injection moulding, mixing, homogenization, cutting, grinding, and compression moulding.

3. Process according to claim 1, wherein a homogenization step (b′) is carried out between steps (b) and (c).

4. Process according to claim 1, wherein the at least one thermoplastic polymer is selected from the group consisting of starches, pure potato amylopectin, polylactic acid, polypropylene, polyethylene, polyhydroxy butyrate, polycaprolactone, polyvinylchloride, polyesters, polyamides, thermoplastic elastomers, natural rubbers, synthetic rubbers for vulcanization, injection mouldable thermoset compounds, and/or any combinations thereof.

5. Process according to claim 1, wherein at least one plasticizer is included in step (b).

6. Process according to claim 1, wherein at least one processing aid is included in step (b).

7. Process according to claim 1, wherein the pre-treatment of cellulose-containing fibers comprises at least one of oxidation, hydrolysis, enzymatic pre-treatment, and/or addition of compounds suitable to swell the cell wall and/or to introduce charges.

8. Process according to claim 1, wherein the cellulose-containing fibers comprised in the granules are substantially non-disintegrated.

9. Granules produced by the process of claim 1.

10. A process for preparing biocomposites comprising microfibrillated cellulose and at least one thermoplastic polymer, said method comprising the steps of:
(a) pre-treating cellulose-containing fibers to facilitate downstream disintegration into microfibrillated cellulose;
(b) adding at least one thermoplastic polymer to the pre-treated cellulose-containing fibers obtained in step (a);
(c) compounding the mixture of step (b) to create granules; and,
(d) processing the granules of step (c) into said biocomposites.

11. A process for preparing biocomposites comprising microfibrillated cellulose and at least one thermoplastic polymer, said method comprising the steps of:
(a) pre-treating cellulose-containing fibers to facilitate downstream disintegration into microfibrillated cellulose;
(b) adding at least one thermoplastic polymer to the pre-treated cellulose-containing fibers obtained in step (a);
(c) processing the mixture of step (b) into biocomposites.

12. The process according to claim 10, wherein the pre-treatment of cellulose-containing fibers comprises at least one of oxidation, hydrolysis, enzymatic treatment, and/or addition of compounds suitable to swell the cell wall and/or to introduce charges.

13. The process according to claim 10, wherein at least one thermoplastic polymer is selected from the group consisting of starches, pure potato amylopectin, polylactic acid, polypropylene, polyethylene, polyhydroxy butyrate, polycaprolactone, polyvinylchloride, polyesters, polyamides, thermoplastic elastomers, natural rubbers, synthetic rubbers for vulcanization, injection mouldable thermoset compounds, and/or any combinations thereof.

14. The process according to claim 10, wherein step (c) is carried out using at least one technique selected from the group consisting of extrusion, injection moulding, mixing, homogenization, cutting, grinding, and compression moulding.

15. The process according to claim 10, wherein step (d) is carried out using at least one technique selected from the group consisting of extrusion, hot-pressing, injection moulding, mixing, homogenization, and compression moulding.

16. A biocomposite produced by the process of claim 10.

17. Granules comprising between approximately 0.1% and 70% pre-treated cellulose-containing fibers and between approximately 30 and 95% of at least one thermoplastic polymer.

18. Granules according to claim 17, further comprising at least one plasticizer.

19. Granules according to claim 17, further comprising at least one processing aid.

20. Granules according to claim 17, wherein the thermoplastic polymer is selected from the group consisting of starches, pure potato amylopectin, polylactic acid, polypropylene, polyethylene, polyhydroxy butyrate, polycaprolactone, polyvinylchloride, polyesters, polyamides, thermoplastic elastomers, natural rubbers, synthetic rubbers for vulcanization, injection mouldable thermoset compounds and/or any combinations thereof.

21. Use of the granules according to claim 17 in a method for producing composites.

22. Use of the granules according to claim 17 in a method for producing composites comprising disintegrated fibers.

23. Use according to claim 21 wherein the method for producing said composites is selected from the group consisting of single-screw extrusion, twin-screw extrusion, triple screw extrusion, and/or injection moulding.
